(19) United States
(12) Patent Application Publication
(10) Pub. No.: US 2022/0037014 A1
(43) Pub. Date: Feb. 3, 2022

(54) Prognostics and Diagnostics of Injection Units and Communications

(71) Applicant: Smart Wires Inc., Union City, CA (US)

(72) Inventors: Aaron Zhou, Fremont, CA (US); Anwar Naseem, Lahore (PK); Zeeshan Khan, Lahore (PK)

(21) Appl. No.: 16/949,604

(22) Filed: Nov. 5, 2020

(30) Foreign Application Priority Data
Jul. 28, 2020 (PK) 494/2020

(51) Int. Cl. G16H 40/67; G16H 50/20; G16H 10/60

(52) U.S. Cl. CPC G16H 40/67; G16H 10/60; G16H 50/20

(57) ABSTRACT

A hierarchical power flow control system includes power flow control devices, a communication coordinator and a gateway. The gateway provides diagnostics, prognostics, and health monitoring, and communicates with an external energy management system (EMS). Operation and actions are based on self-health monitoring, self-prognostics, and analysis and prediction processing in the gateway, in communication with the external energy management system.

### CROSS-REFERENCE TO RELATED APPLICATIONS

This application claims the priority and benefit of Pakistan Patent Application No. 494/2020 filed on Jul. 28, 2020, the disclosure of which is incorporated herein by reference in its entirety.

### TECHNICAL FIELD

This disclosure relates to diagnostics, prognostics, and health monitoring (DPHM) of power flow control devices.

### BACKGROUND

Power flow control systems applied to the grid are complex systems that are difficult to manage and consequently it is challenging to avoid unnecessary shutdowns. Even brief interruptions in service are problematic to both consumers and power utility companies. Such interruptions induce frustration in consumers by seriously compromising their daily activities, or by interrupting business operations. Service interruptions concern power utility companies because of the effect on customers, and because they may stress system components and degrade their lifetime. There is a need in the art for comprehensive and effective monitoring of power flow control equipment, for diagnosing disorders in the equipment, and for prognostics that predict the health of system components with a view to preventive maintenance.

### SUMMARY

In one embodiment, a hierarchical power flow control system is for a power grid. The system has power flow control devices. Each power flow control device has a controller. The controller performs self-health monitoring and self-prognostics of the power flow control device. The power flow control device communicates with a communication coordinator, and the communication coordinator communicates with a gateway. The gateway performs analysis and prediction processing. Operations and actions of the hierarchical power flow control system include diagnostics, prognostics and health monitoring. The hierarchical power flow control system, including the power flow control device, the communication coordinator and the gateway, delivers results to and receives commands from, an energy management system (EMS) coupled to the gateway. The EMS is external to the hierarchical power flow control system. It may reside in a utility control center, operated by utility personnel. It may perform further analysis and prediction processing, based on information provided by the hierarchical power flow control system. The EMS may also receive user input, from which it may produce queries or commands that it communicates to the gateway.

In one embodiment, a method for diagnostics, prognostics, and health monitoring of a distributed power control flow network includes various actions. The method includes communicating regarding self-health monitoring and self-prognostics from power flow control devices to a gateway. The method includes performing analysis and prediction processing at the gateway. The analysis and prediction processing are based on the self-health monitoring and self-prognostics from the power flow control devices. The method includes communicating from the gateway to an EMS regarding the self-health monitoring and self-prognostics, the analysis and the prediction processing. The method may include performing further analysis and prediction processing at the EMS. The method includes operating the plurality of power flow control devices through the distributed power flow control network with diagnostics, prognostics, and health monitoring actions. The operating and the actions are based on the self-health monitoring and self-prognostics of the plurality of power flow control devices, the analysis and prediction processing of the gateway, with user input communicated from the EMS.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 is a schematic diagram showing connectivity between primary system components in an embodiment of the present disclosure.

FIG. 2 depicts an exemplary memory map used for DPHM in an embodiment of the present disclosure.

FIG. 3 depicts an exemplary format of a trigger-based data packet in an embodiment of the present disclosure.

FIG. 4 depicts an exemplary format of a request-based data packet in an embodiment of the present disclosure.

FIG. 5 is a flow chart of an exemplary method for performing prognostics and diagnostics and health monitoring of power flow devices connected by a power flow control network, in an embodiment of the present disclosure.

### DETAILED DESCRIPTION

Diagnostics, Prognostics, and Health Monitoring (DPHM) may be provided in a mesh network of power flow control devices, positioned within and cooperating with a hierarchy of communication and power management subsystems. The power flow control devices in the mesh network may be impedance injection units for example. The hierarchy of communication and power management systems may include for example a communication coordinator having transmitters and receivers for communicating with the power flow control devices, and a gateway for interfacing with the communication coordinator and an EMS having a server within a utility back office. A software platform may provide a framework of rules that apply to algorithms executing across the platform and across the DPHM system. The algorithms may provide continuous monitoring of system health; they may be used to identify device issues; they may also provide failure predictions for scheduling preventive maintenance or shutdown activities.

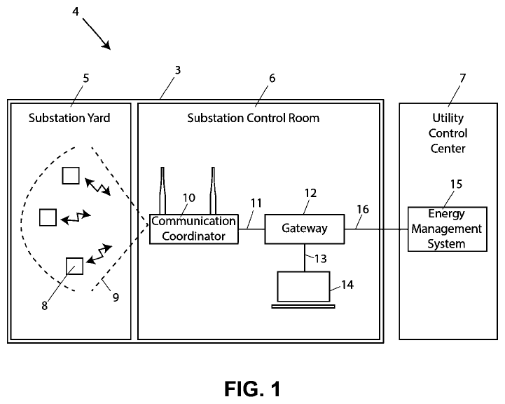

FIG. 1 is a schematic diagram showing connectivity between components and subsystems of a DPHM system 4, in one embodiment. An exemplary DPHM system 4 is distributed across three locations: a substation yard 5, a substation control house 6, and a utility control center 7. Other locations may also be employed, such as when power flow control units are carried on trailers and deployed in a remote location adjacent a tower carrying power transmission lines of the power grid. The power flow control devices 8 may include impedance injection units for example. The impedance injection units may be transformerless (e.g., inverter-based). Each power flow control device 8 may include a suite of sensors and actuators controlled by a controller, thereby providing diagnostic log generation and storage and having capability for self-prognostics and self-health monitoring. The capability may include a low-computational-cost, predictive algorithm for prediction of an imminent failure and sending of any logged data or predictive analysis to a gateway 12 or a DPHM server contained within the EMS for further analysis and potential action.

The devices comprising a hierarchical power flow control system are shown, 3.

Gateway 12 and the DPHM server may each have adequate processing power for detailed analysis and prediction. Each power flow control device 8 may save sensor data at various regular or irregular time intervals, such as but not limited to minutely, hourly, daily, monthly, or yearly. Sensor data may be sent on a scheduled basis, or upon demand or request or event-driven, for example. This data may be retrieved by DPHM system users via gateway 12. The power flow control devices 8 may communicate with each other across a mesh network 9. The mesh network 9 may be an IP (Internet protocol) multimedia subsystem (IMS) standard network, wherein each member of the network can communicate with another member of the network via a proprietary protocol. The DPHM server may be located in the utility control center 7 for example. Network 9 may be controlled by a communication coordinator 10 for example, acting as a communication hub between power flow control devices 8 and a gateway 12. communication coordinator 10 may initiate commands to the local power flow control devices 8 using the wireless mesh network. It may collect status and exchange status information with the gateway 12 via a wired Ethernet connection for example.

Gateway 12 provides a connectivity hub for connecting via a local area network (LAN), link 13, to computer workstations 14 using a universal interface (UI), and via link 11 to the communication coordinator 10. Gateway communication and logging of protocol/data may be logged in gateway 12 and shared with the DPHM server for storage and advanced analysis. DPHM system operators/utility personnel at the computer workstations 14 may interrogate the DPHM system 4, including the DPHM server, to receive monitoring and diagnostic and prognostic information. They may also input directions to the system; for example, these may relate to system configuration or to maintenance issues. In addition, operator instructions may be required for responding to fire emergencies, wherein high winds may require deactivation of power lines for example. These interrogations and instructions may also originate from an Energy Management System (EMS) 15, which maintains the DPHM server. The EMS 15 may be located in the utility control center 7 and may communicate locally, using the local area network, or wirelessly 16 with the gateway 12.

In some embodiments, the power flow control devices 8 are termed Smartwires Field Devices (SWFD), and there are two sub-categories of DPHM at the SWFD level: 1-Trigger-based DPHM, and 2-Request-based DPHM.

Trigger Based DPHM: Whenever any event related to diagnostics, prognostics or health monitoring will take place, an event flag in DPHM memory corresponding to that event will be marked high and consequently a data packet will be sent to the gateway UI.

Request Based DPHM: A computationally expensive prediction algorithm that is not run inside the SWFD so data is sent to the gateway UI where the system has adequate processing power to predict the health of different parameters. A SWFD (e.g., power flow control device 8) in some embodiments should have the capability to save 1 hour, 1 day and 1 month data for selective parameters using an intelligent storage mechanism and transfer such data to gateway (e.g., gateway 12) on request from the gateway UI.

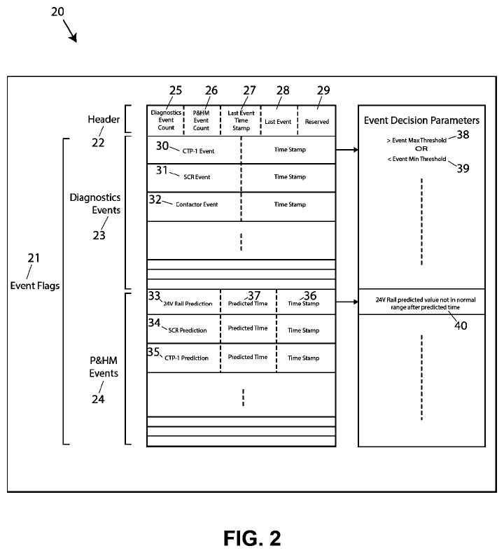

FIG. 2 illustrates an exemplary memory map for a DPHM system (e.g., DPHM system 4 of FIG. 1). In some embodiments, this memory map is accessible from workstation(s) 14 and also accessible from EMS 15. Referring to FIG. 2, memory map 20 may include event flags 21 that may include a header 22, a field for diagnostic events 23, and a field for prognostics and health monitory (P&HM) events 24. The header 22 may include a diagnostics event count 25, a P&HM event count 26, a last event time stamp 27, a last event name 28, and a reserved field 29. Diagnostic events 23 may include current transformer (CTP) events 30, silicon-controlled rectifier (SCR) events 31 which may relate to a bypass of line current during system anomalies (i.e., SCR events 31 may occur when the line current is bypassed during system anomalies), and contactor events 32. A CTP event 30 occurs when a monitored current is outside the bounds of predetermined thresholds. A CTP is used to harvest the power from the primary line current. An extreme low current will cause an event of undervoltage fault, and an extreme high current could cause an open circuit event. Both cases will cause a CTP event 30, which is related to line current. Contactor events 32 relate to contactors, which are mechanical devices. A contactor could fail to open a normally closed switch, or fail to close an already open switch, due to a fault. P&HM events 24 may include a 24V rail prediction 33 when a future failure of the 24V rail becomes more likely, an SCR prediction 34 when failure of one or more SCRs becomes more likely, and a CTP prediction 35 when failure of a current transformer becomes more likely. For each of the P&HM events 24 (e.g., 24V rail prediction 33, SCR prediction 34, CTP prediction 35), a time stamp 36 and a predicted time of failure 37 may be recorded. Time stamp 36 may represent a time of occurrence of the P&HM event (e.g., 24V rail prediction 33, SCR prediction 34, or CTP prediction 35). Predicted time of failure 37 may represent a predicted time when a failure of a particular component (e.g., 24V rail, one or more SCRs, current transformer, etc.) will or is predicted to occur.

The recorded events and predictions feed into a block wherein event decision parameters are calculated. For example, with respect to a diagnostics event 23 (e.g., CTP event 30, SCR event, contactor event), it can be asked: does a measured value exceed an event max threshold 38 or is it smaller than an event minimum threshold 39? With respect to a P&HM event 24 and regarding the 24V rail prediction 33 for example, it can be asked: is the predicted value not in the normal range after a predicted time 40? A measured value for a CTP event 30 could be a line current, for SCT event bypass current under a fault condition, or for a contactor event, a status or report answering: did the contactor open or close as commanded by the software? A predicted value for a 24V rail event would be a predicted voltage of the 24V power supply.

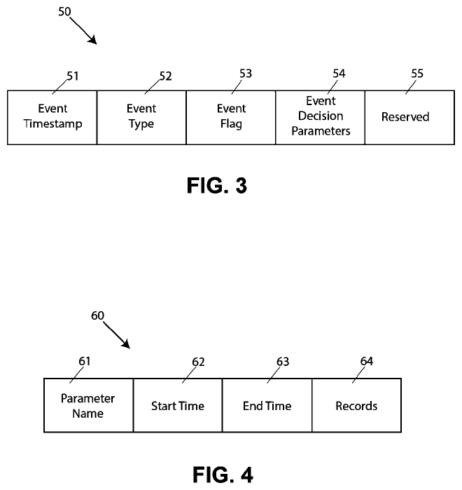

FIG. 3 depicts an exemplary format of a trigger-based data packet in an embodiment of the present disclosure. In FIG. 3, a trigger-based data packet 50 includes an event timestamp 51, an event type 52, an event flag 53, event decision parameters 54, and a reserved field 55. Event timestamp 51 may indicate a time of occurrence of a particular event (e.g., any of the diagnostics events 23 or P&HM events 24 as previously described). Event type 52 may indicate a type of diagnostics event (e.g., CTP event, SCR event, contactor event) or a type of P&HM event (e.g., 24V rail prediction, SCR prediction, CTP prediction). Event flag 53 may indicate whether an event has occurred. For example, when the event (e.g., any of the diagnostics events 23 or P&HM events 24 as previously described) occurs, event flag 53 may hold a TRUE value, and otherwise, event flag 53 may hold a FALSE value, or vice versa. Event decision parameters 54 may include event max threshold 38, event minimum threshold 39, or an event normal range (as previously described).

In one embodiment, trigger-based data packet 50 may be generated in firmware based on certain conditions that result in a recorded event. An example condition would be failure or a prediction of imminent failure of a component, e.g., based on self-health monitoring and self-prognostics of the power flow control devices.

FIG. 4 depicts an exemplary format of a request-based data packet in an embodiment of the present disclosure. Referring to FIG. 4, a request-based data packet 60 includes a parameter name 61, a start time 62, an end time 63, and associated records 64. A request-based data packet may be used for analysis of system components and also for analysis of system behavior with respect to a particular event. In some embodiments, the system is predicting the health of components and tracking various parameters, so that the system can track maintenance requirements or device degradation, for example. One example is measuring temperature of a cooling block associated with IGBT (insulated gate bipolar transistor) switching devices. One parameter, for example, could be IGBT temperature, with a start time of Jan. 1, 2020, end time 2/1/2020, and associated records of 142° C. and 144° C. For consistency, temperature recordings would be associated with line current values, also recorded, since for some components the temperature varies as the line current varies.

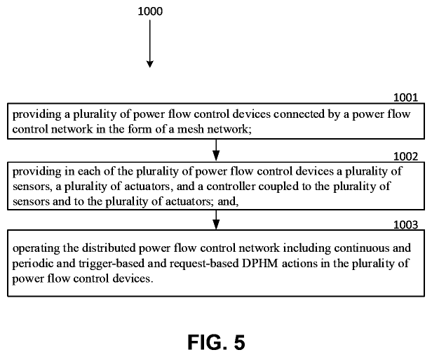

FIG. 5 is a flow chart 1000 of an exemplary method for providing diagnostics, prognostics, and health monitoring (DPHM) of a distributed power flow control network. In step 1001, a plurality of power flow control devices is provided, with the power flow control devices being connected by a distributed power flow control network. The network may be a mesh network. In step 1002, a controller is provided in each of the power flow control devices. In step 1003, the distributed power flow control network is operated, including continuous or periodic or trigger-based or request-based DPHM actions relating to the power flow control devices.

In some embodiments, the DPHM system 4 is a hierarchical system, with the EMS 15 being at the top of the hierarchy, and the power flow control devices 8 at the bottom of the hierarchy. The communication coordinator 10 and the gateway 12 are at mid-level in the hierarchy. DPHM system 4 may include artificial intelligence and deep learning, enabling refinement of the framework of rules and associated actions to be taken. The decision-making timescales may vary from milliseconds for a response to a trigger-based packet for example, to years for a maintenance prediction for example. For greater system reliability DPHM system 4 may include redundant elements, for example a dual gateway 12 connecting with a communication coordinator 10 and/or multiple paths for reliable communication through a mesh network.

The DPHM system 4 implements a framework of rules that is exemplified by the data structures, events, messages, triggers, time stamps, health monitoring, predictions, prognostications and decision parameters described herein.

The foregoing description, for purposes of explanation, uses specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications. They thereby enable others skilled in the art to best utilize the invention and various embodiments with various modifications as are suited to the particular use contemplated. For example, while the detection, mitigation, and elimination of DC component have been illustrated using three-phase power transmission systems, the principles described are equally applicable to any alternating current transmission configuration. That includes two-phase, three-phase, four-phase or any polyphase configuration. The examples are thus illustrative and non-limiting. It is intended that the following claims and their equivalents define the scope of the invention.

What is claimed is:

1. A hierarchical power flow control system for a power grid, comprising:
a plurality of power flow control devices, each configured to inject reactive power into a power transmission line and including a suite of sensors and actuators controlled by a controller for providing diagnostic log generation and storage, self-prognostics and self-health monitoring;
a communication coordinator configured to communicate with each of the power flow control devices, and further configured to communicate with a gateway equipped to perform analysis and prediction processing, including diagnostics, prognostics and health monitoring (DPHM);
wherein user input is directed to the gateway directly, or to an Energy Management System (EMS) residing in an external utility office, wherein the EMS will communicate the user input to the gateway in the form of queries or commands.

2. The hierarchical power flow control system for a power grid of claim 1, further comprising:
a mesh network coupling the plurality of power flow control devices and the communication coordinator.

3. The hierarchical power flow control system for a power grid of claim 1, wherein the communication coordinator coupled to each of the plurality of power flow control devices comprises a mesh network that conforms to an IP (Internet protocol) multimedia subsystem (IMS) standard.

4. The hierarchical power flow control system for a power grid of claim 1, further comprising:
each of the plurality of power flow control devices having one or more sensors and one or more actuators.

5. The hierarchical power flow control system for a power grid of claim 1, wherein the analysis and prediction processing and the further analysis and prediction processing comprise a framework of rules including at least one diagnostic algorithm to identify a disorder or problem by analysis.

6. The hierarchical power flow control system for a power grid of claim 1, wherein the analysis and prediction processing and the further analysis and prediction processing comprise a framework of rules including at least one prognostic algorithm for predicting health of one or more components or subsystems.

7. The hierarchical power flow control system for a power grid of claim 1, wherein the analysis and prediction processing and the further analysis and prediction processing comprise a framework of rules including at least one health monitoring algorithm for monitoring health of one or more components or subsystems.

8. The hierarchical power flow control system for a power grid of claim 1, wherein the plurality of power flow control devices comprises a plurality of impedance injection units.

9. The hierarchical power flow control system for a power grid of claim 1, wherein a decision-making timescale for the energy management system to operate the plurality of power flow control devices varies from milliseconds to years.

10. The hierarchical power flow control system for a power grid of claim 1, further comprising:
a subsystem having a plurality of transmitters and receivers to communicate with a mesh network, wherein the communication coordinator coupled to each of the plurality of power flow control devices comprises the mesh network.

11. The hierarchical power flow control system for a power grid of claim 1, further comprising:
the gateway having connections to a plurality of computer workstations.

12. The hierarchical power flow control system for a power grid of claim 1, wherein the analysis and prediction processing and the further analysis and prediction processing comprise a framework of rules including at least one trigger-based rule.

13. The hierarchical power flow control system for a power grid of claim 1, wherein the analysis and prediction processing and the further analysis and prediction processing comprise a framework of rules including at least one request-based rule.

14. A method for diagnostics, prognostics, and health monitoring (DPHM) of a distributed power flow control network, comprising:
communicating regarding self-health monitoring and self-prognostics from a plurality of power flow control devices to a gateway;
performing analysis and prediction processing at the gateway, based on the self-health monitoring and self-prognostics from the plurality of power flow control devices;
communicating from the gateway to an external energy management system regarding the self-health monitoring and self-prognostics, the analysis and the prediction processing; and
operating the plurality of power flow control devices through the distributed power flow control network with diagnostics, prognostics, and health monitoring actions based on the self-health monitoring and self-prognostics of the plurality of power flow control devices, the analysis and prediction processing of the gateway, supported by communications with an external energy management system.

15. The method of claim 14, further comprising:
performing, in one or more of the plurality of power flow control devices, at least one diagnostic algorithm for identification of a disorder or a problem by analysis.

16. The method of claim 14, further comprising:
performing, in one or more of the plurality of power flow control devices, at least one prognostic algorithm for predicting health of one or more components or systems.

17. The method of claim 14, further comprising:
performing, in one or more of the plurality of power flow control devices, at least one health monitoring algorithm for monitoring health of one or more components or systems.

18. The method of claim 14 wherein the diagnostics, prognostics, and health monitoring actions comprise continuous, periodic, trigger-based, or request-based DPHM actions.

19. A method for diagnostics, prognostics, and health monitoring (DPHM) of a distributed power flow control network, comprising:
providing a plurality of power flow control devices connected by a power flow control network in the form of a mesh network;
providing in each of the plurality of power flow control devices a plurality of sensors, a plurality of actuators, and a controller coupled to the plurality of sensors and to the plurality of actuators; and
operating the distributed power flow control network including continuous and periodic and trigger-based and request-based DPHM actions in the plurality of power flow control devices.
